(19) United States
(12) Patent Application Publication
(10) Pub. No.: US 2022/0146861 A1
(43) Pub. Date: May 12, 2022

(54) METHOD FOR DETERMINING THE OPTICAL CENTER POINT OF THE LENSES OF SPECTACLES TO BE MANUFACTURED FOR A SPECTACLE WEARER

(71) Applicant: Silhouette International Schmied AG, Linz (AT)

(72) Inventors: Stephan Lechner, Linz (AT); Rupert Spindelbalker, Puchenau (AT)

(73) Assignee: Silhouette International Schmied AG, Linz (AT)

(21) Appl. No.: 17/432,524

(22) PCT Filed: Jan. 21, 2020
(86) PCT No.: PCT/AT2020/060013

§ 371 Date: Aug. 20, 2021

(30) Foreign Application Priority Data
Feb. 21, 2019 (AT) A 50141/2019

(51) Int. Cl. G02C 13/00; G06T 7/73; G06T 7/60; H04N 5/272; A61B 3/11; A61B 3/00

(52) U.S. Cl. CPC G02C 13/005; G06T 7/73; G06T 7/60; H04N 5/272; G06T 2207/30204; A61B 3/0041; G06T 2207/30201; G06T 2207/30041; A61B 3/111

(57) ABSTRACT

A method and a device are described for determining the optical center point of the lenses of spectacles to be manufactured for a spectacle wearer, whose head is imaged from the front using a digital camera (10), wherein the location of the pupils (16) and the interpupillary distance (20, 21) are determined with the aid of an image evaluation program to define the optical center point of the lenses. To provide advantageous conditions, it is proposed that the face of the spectacle wearer is imaged having a measurement frame (1) held on the head via a nose rest (4) and earpieces (3) and having a view oriented on a fixation point (13) fixedly associated with the camera (10), a front region of the measurement frame (1) has two lateral marking points (7) and a middle marking point (8) placed frontally in front of these lateral marking points (7), the angles of the viewing lines (15) in relation to the image plane are determined with the aid of the image evaluation program from the mutual distances and locations, which are measured in the facial image, of the pixels of the three marking points (7, 8) and the pupils (16), before the measurement points (19), which are corrected with respect to parallel viewing lines (17), are ascertained to determine the interpupillary distances (20, 21) in relation to the nose rest (4).

### TECHNICAL AREA

The invention relates to a method and a device for determining the optical center point of the lenses of spectacles to be manufactured for a spectacle wearer, whose head is imaged from the front using a digital camera, wherein the location of the pupils and the interpupillary distance are determined to define the optical center point of the lenses with the aid of an image evaluation program.

### PRIOR ART

To be able to fit spectacles to spectacle wearers before they are manufactured, recording the head of the spectacle wearer while wearing spectacles by way of a middle digital camera and two lateral digital cameras is known (US 2018/0005448 A1), in order to calculate a three-dimensional model of the head with spectacles with the aid of the recorded images. The spectacles worn by the spectacle wearer can be suppressed from the displayed two-dimensional facial image with the aid of a display screen displaying the facial image of the middle camera on the basis of the acquired data and can be replaced by the image of spectacles selected from various models, which are imaged in an alignment corresponding to the spectacles digitally recorded with the head of the spectacle wearer and overlaid on the facial image. With the aid of the three-dimensional recording of the head, in addition the interpupillary distance required for determining the optical center point of the lenses of the spectacles to be selected can be ascertained, specifically in consideration of a possible angle between the viewing lines of the two eyes. This is because the rotational angle of the eyeballs can be acquired via the location of the iris between the eyelids. This and similar known methods for fitting spectacles yet to be manufactured to the anatomical conditions of a spectacle wearer have the disadvantages not only of the significant computing effort to create a digital, three-dimensional head model but also the circumstance that the arrangement of the eyeballs in relation to the head and the interpupillary distance can be ascertained with sufficient accuracy, but the location of the pupils in relation to the spectacle lenses of the spectacles to be selected cannot be determined.

### DESCRIPTION OF THE INVENTION

The invention is therefore based on the object of designing a method for determining the optical center point of the lenses of spectacles to be manufactured for a spectacle wearer so that the optical center point of the lenses of the spectacles to be manufactured can be determined with high accuracy using comparatively little computing effort, and with a low handling effort.

Starting from a method of the type described at the outset, the invention achieves the stated object in that the face of the spectacle wearer is imaged having a measurement frame held via a nose rest and earpieces on the head with a view oriented on a fixation point fixedly associated with the camera, a front region of the measurement frame has two lateral marking points and a middle marking point placed frontally forward from these two lateral marking points, the angles of the viewing lines in relation to the image plane are determined with the aid of the image evaluation program from the mutual distances of the pixels of the three marking points and the pupils measured from the two-dimensional digital facial image, before the measurement points corrected with respect to parallel viewing lines are ascertained to determine the interpupillary distances in relation to the nose rest.

By putting on a measurement frame, which is supported like spectacles via a nose rest and earpieces on the head of a spectacle wearer, the location of this measurement frame in relation to the pupils of the two eyes can be acquired with the aid of marking points on the measurement frame by a merely two-dimensional digital facial image, under the condition that the distance of the front region of the measurement frame between the two earpieces is approximately equal to the two eyes, which has to be presumed in the case of a good spectacle seat.

The at least three marking points in the front region of the measurement frame, of which the middle is placed frontally forward from the two lateral marking points, ensure that in a digital facial image, on the basis of the mutual locations and distances resulting from the pixels of these marking points, the distance and the location of the camera in relation to these marking points and thus in relation to the measurement frame can be ascertained, because due to the predetermined mutual geometrical association, these marking points may be imaged in a unique dependence on the alignment of the optical axis and the distance of the objective from the markings in the image plane. This means that in the two-dimensional facial image, due to the alignment of the image plane in relation to the measurement frame, the pixels of the pupils are also aligned in relation to the measurement frame.

However, it cannot be presumed that the viewing lines of the two eyeballs extend in parallel. The face is therefore recorded with eyes focused on a fixation point associated with the camera, so that due to this focusing, the viewing lines of the two eyes enclose angles with the image plane, so that the image distance of the two pupils does not correspond to the interpupillary distance with parallel viewing lines. Since in a frontal facial image, the image plane extends at least approximately parallel to the front region of the measurement frame and possibly the pixels can be corrected in this regard on the basis of the acquired alignment of the camera in relation to the measurement frame, the viewing lines can be aligned in parallel to one another by a corresponding rotation around the rotation points of the eyeballs, which requires a corresponding displacement of the pixels of the pupils in the image plane, so that measurement points corrected by this displacement of the pixels of the pupils are available for determining the interpupillary distance from the two-dimensional facial image. The corrected pixels of the pupils in relation to the measurement frame are also uniquely defined using the alignment of the measurement frame in relation to the nose caused by the nose rest. The corrected pixels of the pupils thus determine the optical center point of spectacle lenses which are held in a spectacle frame supported, corresponding to the measurement frame, via a nose rest and earpieces.

Particularly simple handling conditions result for the method according to the invention if the two-dimensional facial image recorded by the digital camera is displayed on a display screen associated with the camera, because in this case not only the coordinates of the optical center point for the lenses to be manufactured of spectacles can be easily displayed, but also the condition is created in which, on the facial image displayed on the display screen, the suppressed measurement frame is overlaid with an image of spectacles aligned corresponding to the measurement frame, so that the spectacles to be manufactured are visible in the facial image on the display screen, wherein the optical center points for the lenses result directly by way of the corrected measurement points of the pupils.

The fitting of the image of the spectacles to be manufactured to the supports predetermined by the measurement frame via the nose rest and the earpieces require not only a frontal, but also a lateral acquisition of the measurement frame via corresponding marking points. For this purpose, a digital lateral image of the head with measurement frame put on can be created using the camera, which measurement frame is provided with at least one additional marking point associated with an earpiece for the location acquisition of the earpieces.

To carry out such a measurement method, it is possible to start from a device having a digital camera and having an image evaluation unit connected to a display screen for digital facial images recorded by the camera. In this case, a measurement frame having a nose rest and earpieces for support on the head of the spectacle wearer having at least three marking points arranged in the frontal region of the measurement frame, namely two lateral marking points and one middle marking point placed frontally forward from these lateral marking points, and an image evaluation unit are to be provided, which, from a facial image acquired by the camera having measurement frame put on, ascertains the angles of the viewing lines in relation to the image plane and calculates therefrom measurement points corrected with respect to parallel viewing lines for the determination of the interpupillary distance from the pixels of the marking points and the pixels of the pupils aligned on a fixation point associated with the camera.

Particularly simple design conditions result if the measurement frame has the form of semi-rimless spectacles, which facilitates the replacement in the facial image of the measurement frame by spectacles, whose association with the head of the spectacle wearer is based on a support on the head of the spectacle wearer corresponding to the measurement frame. For this purpose, however, the location of the spectacle earpieces is also to be acquired, which is possible in a simple manner in that the measurement frame has at least one further marking point in the region of the earpieces, so that the seat of the measurement frame can be defined using a second lateral head image.

### BRIEF DESCRIPTION OF THE INVENTION

The subject matter of the invention is illustrated by way of example in the drawings. In the figures

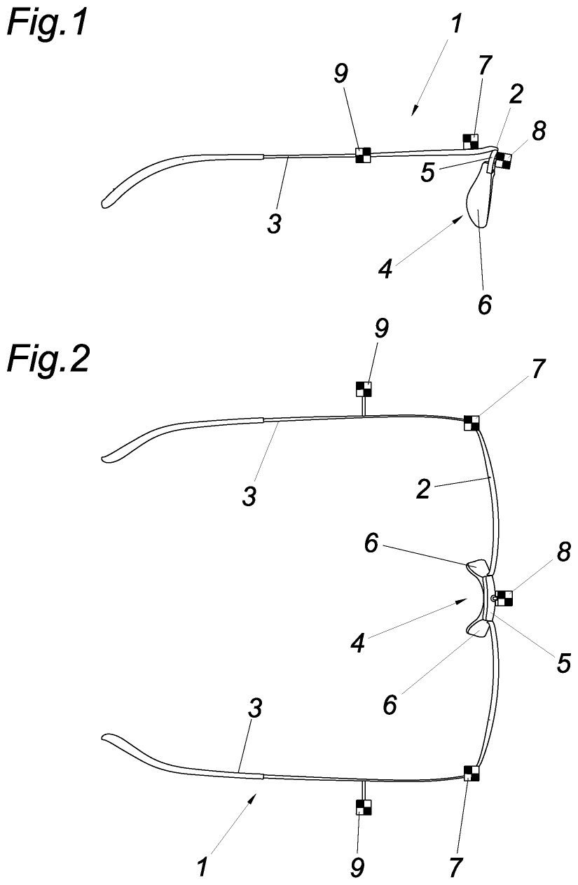

FIG. 1 shows a measurement frame used in the method according to the invention in a side view,

FIG. 2 shows the measurement frame of FIG. 1 in a top view, and

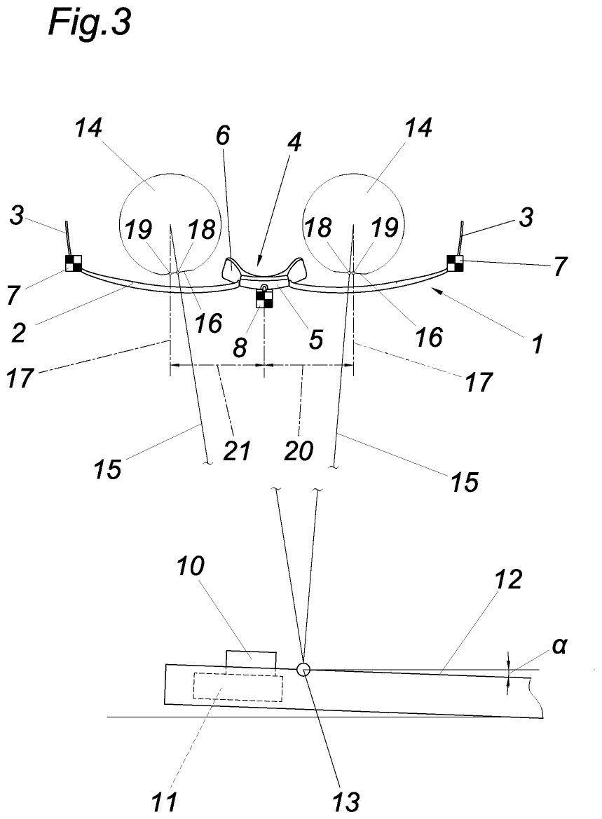

FIG. 3 shows a schematic illustration of a measurement arrangement according to the invention.

### WAYS OF EMBODYING THE INVENTION

The measurement frame 1 used has a form corresponding to semi-rimless spectacles without lenses and comprises a front region 2 having two earpieces 3. The front region 2 is provided with a nose rest 4, which can be formed in a typical way from support cushions 6 supported by a nose bridge 5. The measurement frame 1 is therefore supported on the head like spectacles via the nose rest 4 and the earpieces 3. To determine the location, the measurement frame 1 is equipped in the front region 2 with two lateral marking points 7 and with a middle marking point 8. The arrangement is made here so that the middle marking point 8 is associated with the nose bridge 5, but the two lateral marking points 7 are arranged in the transition region from the front region 2 to the earpieces 3, and are set back in relation to the middle marking point 8 in the frontal direction. In addition, the earpieces 3 are provided with additional marking points 9, which facilitates acquiring the location of the measurement frame 1 in space. For better optical acquisition of the marking points 7, 8, 9, they have a cube shape having high-contrast centering fields.

To be able to determine the optical center point of the lenses of spectacles to be manufactured for a spectacle wearer beforehand, according to FIG. 3, the head of the spectacle wearer with measurement frame 1 put on is frontally recorded with the aid of a digital camera 10. The obtained digital two-dimensional facial image shows the marking points 7 and 8 required for acquiring the location of the measurement frame 1 in a mutual relative location defined by the projection in the direction of the axis of the objective of the camera 10, so that on the basis of the knowledge of the mutual geometric association of these marking points 7, 8 and the optical data of the objective of the camera 10, the distance and alignment of the image plane in relation to the measurement frame 1 can be calculated by means of an image evaluation unit 11 associated with the camera 10 from the mutual distances and the mutual location of the pixels of these marking points 7, 8 in the two-dimensional facial image.

According to FIG. 3, the camera 10 is assembled with the image evaluation unit 11 and a display screen 12 for displaying the digital facial image to form a shared handling unit, as is known, for example, in the form of mobile telephones. The facial image displayed on the display screen 12 can also be used to replace the suppressed measurement frame 1 by way of the image of spectacles available for selection, specifically in the location predetermined by the measurement frame 1 and determined via the nose rest 4 and the earpieces 3 in relation to the head of the spectacle wearer.

Since the viewing lines of the eyes oriented straight ahead into the distance are to extend through the optical center points of the lenses and such an eye alignment cannot be expected from the spectacle wearer, a fixation point 13 is associated with the camera, which in no way needs to be formed by the objective of the camera 10. In FIG. 3, this fixation point 13 to be focused on by the spectacle wearer is displayed, for example, on the display screen 12. Due to this focusing on the fixation point 13, viewing lines 15 which each include an angle with the image plane result between the rotation point of the eyeballs 14 and the fixation point 13. This image plane coincides with the display screen plane in this exemplary embodiment, but this is not required.

The alignment of the pupils 16 caused by focusing on the fixation point 13 results in an incorrect determination of the interpupillary distance, however, because parallel viewing lines are decisive for the determination of the optical center points of the lenses, as are indicated by dot-dash lines and provided with the reference sign 17. The pupil location therefore has to be corrected in accordance with the angles of the viewing lines to be able to enter the measurement points 19 corrected in relation to the recorded pupil location 18 to determine the interpupillary distance in the two-dimensional facial image.

During the recording of the face of the spectacle wearer, the image plane of the camera 10 is only approximately aligned in parallel to the front region 2 of the measurement frame 1. To avoid location errors of the corrected measurement points 19 thus caused, a corresponding inclination a of the image plane in relation to the frontal plane of the measurement frame 1 can be taken into consideration. This inclination a results due to the alignment of the objective axis of the camera 10 in relation to the measurement frame 1 and is determined by the image evaluation of the marking points 7, 8.

Using the corrected measurement points 19, the left and right interpupillary distances 20, 21 in relation to the measurement frame 1 and thus the optical center points for lenses may be determined with high accuracy and comparatively low effort, independently of the type of the lenses to be manufactured, because the location of the spectacles to be fitted in relation to the head is predetermined by the measurement frame, so that the parallel viewing lines 17 define the optical center points for the lenses of each pair of spectacles to be manufactured.

1. A method for determining the optical center point of the lenses of spectacles to be manufactured for a spectacle wearer, whose head is imaged from the front using a digital camera (10), wherein the location of the pupils (16) and the interpupillary distance (20, 21) are determined with the aid of an image evaluation program to define the optical center point of the lenses, characterized in that the face of the spectacle wearer is imaged having a measurement frame (1) held on the head via a nose rest (4) and earpieces (3) and having a view oriented on a fixation point (13) fixedly associated with the camera (10), a front region of the measurement frame (1) has two lateral marking points (7) and a middle marking point (8) placed frontally in front of these lateral marking points (7), the angles of the viewing lines (15) in relation to the image plane are determined with the aid of the image evaluation program from the mutual distances and locations, which are measured in the facial image, of the pixels of the three marking points (7, 8) and the pupils (16), before the measurement points (19), which are corrected with respect to parallel viewing lines (17), are ascertained to determine the interpupillary distances (20, 21) in relation to the nose rest (4).

2. The method according to claim 1, characterized in that the two-dimensional facial image recorded by the digital camera (10) is displayed on a display screen (12) associated with the camera (10).

3. The method according to claim 2, characterized in that, in the facial image displayed on the display screen (12), the suppressed measurement frame (1) is overlaid with an image of spectacles aligned corresponding to the measurement frame (1).

4. The method according to any one of claims 1 to 3, characterized in that a digital lateral image of the head with measurement frame (1) put on is created using the camera (10), which measurement frame (1) is provided with at least one additional marking point (9) associated with an earpiece (3) for the location acquisition of the earpieces (3).

5. A device for determining the optical center point of the lenses of spectacles to be manufactured for a spectacle wearer, having a digital camera (10) and having an image evaluation unit (11) connected to a display screen (12) for digital facial images recorded by the camera (10), characterized in that a measurement frame (1) having a nose rest (4) and earpieces (3) for support on the head of the spectacle wearer is provided with at least three marking points (7, 8) arranged in the front region (2) of the measurement frame (1), namely two lateral marking points (7) and a middle marking point (8) placed frontally in front of these lateral marking points (7), and the image evaluation unit (11) ascertains the angles of the viewing lines (15) in relation to the image plane from a facial image having measurement frame (1) put on, which is acquired by the camera (10), from the pixels of the marking points (7, 8) and the pixels of the pupils (16) focused on a fixation point (13) associated with the camera (10) and calculates measurement points (19) corrected with respect to parallel viewing lines (17) for the determination of the interpupillary distance (20, 21) therefrom.

6. The device as claimed in claim 5, characterized in that the measurement frame (1) has the form of semi-rimless spectacles.

7. The device as claimed in claim 5 or 6, characterized in that the measurement frame (1) has at least one further marking point (9) in the region of the earpieces (3).
